(19) United States
(12) Patent Application Publication
(10) Pub. No.: US 2010/0004330 A1
(43) Pub. Date: Jan. 7, 2010

(54) ANTI-OXIDATIVE CONTENT MATERIAL USED IN DRINK AND FOOD MANUFACTURING METHOD

(76) Inventors: Li Lin Huang, Sinjhuang City (TW); Chris IngYi Huang, Sinjhuang City (TW)

Correspondence Address: SINORICA, LLC, 2275 Research Blvd., Suite 500, ROCKVILLE, MD, 20850, US

(21) Appl. No.: 12/166,519

(22) Filed: Jul. 2, 2008

(51) Int. Cl. A61K 31/375

(52) U.S. Cl. 514/474

(57) ABSTRACT

The present invention relates to a food or drink which provides anti-ageing, anti-cancer, anti-heart disease, vision-enhancing effects of antioxidant substances. This method contains the following anti-oxidative supplements, and is added in food or drink in certain ratio. The method is as follows: adding into a fixed quantity of food or drink an appropriate quantity of the following effective content: vitamin C (water-soluble), β-carotene (lipid-soluble), lycopene (lipid-soluble), lutein (lipid-soluble) and surfactants. These supplements do not result in mutual interference of absorption and/or biological effect but are more effective in reducing free-radicals.

### BACKGROUND OF THE INVENTION

1. Field of the Invention

The present invention relates to a food or drink which provides anti-ageing, anti-cancer, anti-heart disease, vision-enhancing effects of antioxidant substances; especially, the present invention relates to a food or drink which has lipid-soluble carotenoids mixed with water-soluble vitamin C and relates to food and drink manufacturing method thereof.

2. Background Art

At present, intake of all kinds of antioxidants in human body must depend upon the ingestion of natural foods, health foods, or medicine. This anti-oxidation cannot achieve optimal anti-aging, anti-cancer, anti-heart disease, vision-enhancing effects.

It is known that many antioxidants are of benefit to human body. For example:

(1) Lutein, which belongs to lipid-soluble carotenoids group, exists naturally in green spinach and cabbage. In 1994, Harvard University researchers found that a person ingesting 6 mg per day of lutein lead to a 43% lower risk for macular degeneration (Seddon JM 1994)

Its molecular formula is C₄₀H₅₆O₂, and the structural formula is:

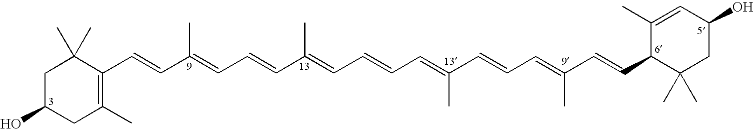

It has an obvious beneficial effect in the improvement of Age-related Macular Degeneration (AMD) (Landrum JT 1997).

If such diseases (AMD) are not prevented or cured, they could cause sandblindness. Thus a straight line could appear to look like a bending deformation. A blind spot could gradually form in the central part of vision in a more serious case.

UV rays can generally be filtered out by the cornea and lens, but the blue light may pass through the eyeball and directly reach the retina and macula lutea so as to produce large quantities of free radicals which cause cataracts and macular degeneration. Lutein can filter out blue light to avoid damage of the eye by the said blue light. Apart from its presence in the macular area, lutein also exists in the other tissues (the lens, the iris and the ciliary body) of the eyes and plays an important role in the preservation of the health of the eyes.

Lutein is a good antioxidant. It is able to neutralize the free radicals and prevent cell damage by free radicals. Lutein inside the serum can reduce the blood vessel wall thickening. (Dwyer J H 2001)

Lutein is originally a carotenoid which exists in the natural fruits and vegetables. The human body cannot synthesize lutein and must obtain lutein from food.

(2.) Lycopene is an unsaturated lipid-soluble hydrocarbon in a straight chain. Lycopene is most stable in the nature and exists in trans-form. However, external factors such as light, heat, acid, oxygen or other chemical reagents can isomerize lycopene to form cis-form. Most lycopenes separated from the plant are of trans-form, but said lycopene in human plasma exists equally in 50% trans- and 50% cis-form (Stahl W. 1992, Schierle J 1997). This indicates that cis-lycopene is more easily absorbed by the human body than trans-lycopene. Lycopene bioavailability in processed tomato products is higher than in unprocessed fresh tomatoes. (Shi J 2000) Its molecular formula is C₄₀H₅₆, and its structural formula is as follows:

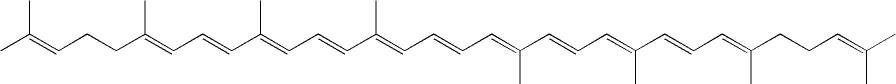

Many epidemiologic studies have found that the increased intake of tomato products, as well as elevated lycopene content in blood is inversely proportional to cancer risk. This is strong evidence for lycopene's anti-carcinogenic activity. In a 1999 literature review by Giovannucci E. among 72 studies identified, 57 reported inverse associations between tomato intake or blood lycopene level and the risk of cancer at a defined anatomic site; 35 of these inverse associations were statistically significant. The beneficial effect of increased tomato intake or increased blood lycopene level is especially observed in prostate, lung and gastric cancers. The beneficial effect is also observed in rectal cancer (Freudenheim J L 1990), esophageal, oral cancers (La Vecchia C 2002), and pancreatic cancer (André Nkondjock 2005). Additionally, the beneficial effect is observed in the cervical cancer (VanEenwyk J 1991).

Lycopene's preventive effects in prostate cancer (Gann P H 1999), and coronary heart disease (Rao A V 2002) have been reported in the literatures.

Based on a multicenter, multinational EURAMIC (European community multicenter study on antioxidants, myocardial Infarction, and breast cancer) clinical study, lycopene has been shown to be independently protective of myocardial infarction, with an odds ratio of 0.52 for the contrast of the 10^{th} and 90^{th} percentiles of carotenoid level in adipose tissue (Kohlmeier L 1997).

/(3) Beta-carotene (β-carotene) is a powerful lipid-soluble antioxidant. Beta-carotene has a molecular formula of C₄₀H₅₆, and its structural formula is as follows:

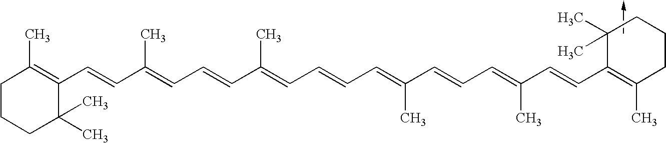

β-carotene can reduce by 45% the risk of cardiovascular disease among the middle-aged or elderly (Klipstein-Grobusch K 1999). The antioxidant beta-carotene has been implicated in preventing or slowing down the atherosclerotic process by inhibiting LDL oxidation (Reaven P D 1996).

It can also contribute to the protection of lung function (Guénégou A 2006).

(4.) Besides the above functions, β-carotene, lutein and lycopene can also prevent arteriosclerosis.

Tissue factor exists in the tissue, platelets and leukocytes. It is a plasma protein which is involved in the blood coagulation cascade. Upon vascular injuries, tissue factor is elevated to initiate blood coagulation. The increase in radicals in the vascular wall will lead to an increase in tissue factor.

Dong Kun Lee and others (2006) published that formation and increase of tissue factor are related to development of cardiovascular disease. Due to an increase of free radicals inside vascular wall, tissue factor will increase the activity of endothelial cells so as to lead to the blood clots (thrombosis of blood vessels). The experimental study of all carotenoids find that once β-carotene, lutein, and lycopene are respectively added into micro-emulsion, the change in their water-solubility could suppress tissue factor activity (P<0.01).

Effectiveness of different concentrations of carotenoids to the activity of tissue factor of internal cells is shown in FIGS. 1, 2 and 3.

### BRIEF DESCRIPTION OF DRAWINGS

FIG. 1 is showing an effectiveness of different concentrations of β-carotenoids to the activity of tissue factor of internal cells.

FIG. 2 is showing an effectiveness of different concentrations of Lutein to the activity of tissue factor of internal cells.

FIG. 3 is showing an effectiveness of different concentrations of Lycopene to the activity of tissue factor of internal cells.

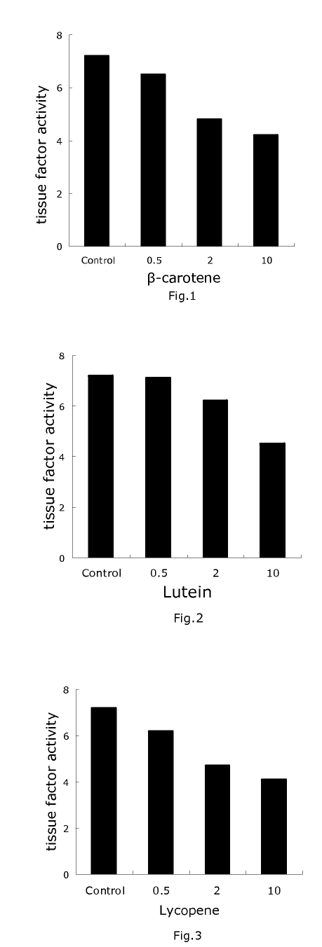

Effects of various concentrations of carotenoids on tissue factor activity. Carotenoids used were 0.5, 2 and 10 μmol/L of β-carotene, lutein and lycopene. Control is a microemulsion without carotenoids.

It is known that these three types of carotenoids will suppress the activity of tissue factor, which is strong evidence that they can prevent the occurrence of cardiovascular disease.

(5) Vitamin C is a water-soluble antioxidant able to protect cells and enhance the activities of leucocytes. Vitamin C is concerned with enhancing immunity, solidifying tissue, and capturing free radicals. It is able to prevent nitrite from combining with amine to form the carcinogenic products of ammonium nitrite. Thus, Vitamin C is one of the anti-carcinogenic nutrients. Vitamin C plays an important role in physiological repair when the tissue is injured. Lack of Vitamin C will result in slow wound healing. Vitamin C can effectively scavenge stress-induced reactive oxygen and will be consumed greatly under oxidative stress. Therefore, Vitamin C should be added in large quantity. Its molecular formula: C₆H₈O₆, the structural formula is as follows:

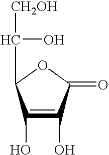

### SUMMARY OF THE INVENTION

In view of the above, the main purpose of the present invention is to provide a drink or food that will provide lipid-soluble carotenes such as β-carotene, lycopene, lutein and water-soluble antioxidants such as Vitamin C.

Another purpose of the present invention is to provide a food or drink which can have anti-aging, anti-cancer, anti-heart disease, vision-enhancing effect on human body and provide the maximal benefit and the best results for the said human body.

### DETAILED DESCRIPTION OF INVENTION

The mixture(s) of four antioxidants can synergistically reduce oxidative damage resulting from normal cell functions and outside sources to a minimum. Three lipid-soluble carotenoids of lutein, lycopene, and β-carotene mixed supplements have been confirmed to reduce lymphocyte DNA internal bond breakage and DNA damage (Pool-Zobel B L, Carcinogenesis 1997; Cancer Epidemiol Biomarkers Prev. 1998). The present invention has been shown to strengthen the antioxidant effect after the water-soluble vitamin C is added. The four antioxidants act synergistically and complementarily exert effect on other antioxidants.

Although there exist other types of antioxidants, long-term studies have shown that too many types of antioxidants or an excessive amount of antioxidants will result in the mutual interferences between the absorption in the human body and the biochemical effect on the human body.

The elderly and adults living in the complex industrial society, and under the pressure of the working environment (which is the situation most adults encounter daily) and those who are exposed to UV-radiation, toxic chemicals, infectious organisms or other strongly harmful agents, will possibly suffer from more oxidative damage (due to the changes from the internal activities and external agents). In addition, a variety of antioxidants used to strengthen the anti-oxidative effect in the human body is believed more likely to help the body prevent disease and stress-induced oxidation whereby to avoid the cell damage.

A study has shown that ingestion of a combined dose of Beta-Carotene and lycopene improves the absorption of lycopene than when lycopene is taken alone (Johnson E J 1997).

With the proportion and the number of doses within the claimed scope of the present invention, the tests of the above mentioned four antioxidants have shown that in the lowest dose and in an appropriate proportion, they can protect the body most effectively, as well as reduce the amount of damage to DNA. Furthermore, for each antioxidant within the claimed scope of the present invention, when its amount is increased from 1.5 times to 2 times, its effectiveness is still very good.

Antioxidants materials used in the present invention are Vitamin C, β-carotene, lycopene, and lutein produced by fine chemical synthesis or extraction from natural food. Furthermore, it is made into water-soluble or lipid-soluble powder through special drying treatment, then manufactured into dubbed powder dose (or added a tracing amount of surfactants) or formed into a tablet for the users.

Oxidative stress in the cellular environment results in the formation of highly reactive and unstable lipid hydroperoxides. Decomposition of the unstable peroxides derived from polyunsaturated fatty acids results in the formation of malondialdehyde (MDA) which can be quantified calorimetrically following its controlled reaction with thiobarbituric acid. The Thiobarbituric Acid Reactive Substances (TBARS) assay is used for screening and monitoring lipid peroxidation, a major indicator of oxidative stress.

TBARS method is an indicator used by most scholars to determine the free radical level resulting from human disease (illness) or tissues injury. The higher antioxidant activity results in lower concentration of thiobarbituric acid-reactive substances (TBARS).

Stahl W, in 1998, used TBARS method to measure antioxidant activity of single and mixed carotenoids respectively in reducing free radicals. The results are shown in following table:

single carotenoid

| Sample | TBARS (%) measured |
|---|---|
| Control | 100 ± 9 |
| Lycopene | 25 ± 10 |
| β-carotene | 73 ± 9 |
| Lutein | 77 ± 9 |

Control: no antioxidants.

mixed carotenoid

| Name of Sample | TBARS (%) measured |
|---|---|
| Lycopene plus β-carotene | 31 ± 10 |
| Lycopene plus Lutein | 16 ± 8 |
| Lutein plus β-carotene | 44 ± 8 |

Control: no antioxidants.

As shown in the above two tables, the antioxidant activity of lycopene is the highest, followed by the antioxidant activities of Beta-carotene and lutein. Mixed carotenoids are more effective in preventing harm from oxidative stress than a single carotenoid. Most obviously, a mixture of lycopene plus lutein will greatly improve its antioxidant effect.

According to the present invention, the products of the Beta-carotene, lutein, lycopene, and of mixture of these three (in 0.5:4:4 optimal weight ratio) from the Allied Biochemical Industrial Co., Ltd were sent to “Food Industrial Research And Development Institute” (331 Shih-Pin Road, Hsinchu, 300 Taiwan) for antioxidant activity testing (DPPH scavenging activity). The data are shown in the following table:

| | Name of Article | Scavenging rate/g | Report No. |
|---|---|---|---|
| 1. | β-carotene | correspond to 7.08 × 10³ ppm VitE | 095SA02236 |
| | | Scavenging rate/g | |
| 2. | Lycopene | correspond to 2.35 × 10⁴ ppm VitE | 095SA02233 |
| | | Scavenging rate/g | |
| 3. | Lutein | correspond to 7.69 × 10² ppm VitE | 095SA02235 |
| | | Scavenging rate/g | |
| 4. | Mixtures of 1, 2 | correspond to 5.48 × 10⁴ ppm VitE | 095SA02349 |
| | and 3 | Scavenging rate/g | |

It can be observed from the above table that a free radical scavenging rate of the mixture of the present invention equals to 5.48×10⁴ ppm VitE, which is 2.3 times the rate of lycopene, 7.8 times the rate of β-carotene, and 71.26 times the rate of lutein. The hierarchy of its antioxidant activity is: mixture>lycopene>β-carotene>lutein. It shows that the mixture of the present invention exactly match the above Stahl's disclosure that “a mixture of the two will substantially increase its antioxidant effect” and have a more synergistic effect, its antioxidant activity is greater than all the single antioxidant activity.

### EXAMPLES

In order to elucidate the present invention, the descriptions are made further by way of the following examples:

### Example 1

Subjects were 23 healthy and non-smoking males aged from 27 to 40. During the study, the subjects ate normal diets but abstained from vegetables containing high units of carotenoids. After a 2 week depletion period, they drank daily 330 ml tomato juice with 40 mg lycopene (weeks 3 and 4), 330 ml carrot juice with 22.3 mg β-carotene and 15.7 mg α-carotene (weeks 5 and 6), and 10 g dried spinach powder (in water or milk) with 11.3 mg lutein (weeks 7 and 8).

In the course of the study, blood was collected weekly. The “COMET” assay was used to detect DNA damage in peripheral blood lymphocytes. The supplementation of the diet with tomato, carrot, or spinach products resulted in a significant decrease in endogenous levels of strand breaks in lymphocyte DNA. Oxidative base damage was significantly reduced during the carrot juice intervention. These findings support the hypothesis that carotenoid containing plant products exert a cancer-protective effect via a decrease in oxidative and other damage to DNA in humans. (Pool-Zobel B L et al., Carcinogenesis. 1997 September;18(9):1847-50 )

### Example 2

A randomized, double-blind, placebo-controlled interventional study was conducted. 37 healthy, nonsmoking females whose post-menopausal ages were between 50 and 70 years were assigned to one of five groups randomly and were instructed to consume a daily dose of mixed carotenoids (β-carotene, lycopene, and lutein; 4 mg each), 12 mg of a single carotenoid (β-carotene, lycopene, or lutein), or placebo for 56 days.

Plasma carotenoid concentrations were analyzed by using HPLC, and lymphocyte DNA damage was measured by using a single-cell gel electrophoresis (comet) assay.

Results: At day 57, all carotenoid-supplemented groups showed significantly lower endogenous DNA damage than at baseline (P<0.01), whereas the placebo group did not show any significant change. Significantly less (P<0.05) endogenous DNA damage was found as early as day 15 in the mixed carotenoid (P<0.01) group.

Conclusions: The results indicate that carotenoid supplementation decreases DNA damage and that a combination of carotenoids (4 mg each of lutein, β-carotene, and lycopene), an intake that can be achieved by diet, or a large dose (12 mg) of individual carotenoids exerts protection against DNA damage. (Zhao X et al., Am J Clin Nutr. 2006 January;83(l):163-9)

### Example 3

Standard diet was given to 20 healthy women with average age of 25.2. Subjects were instructed to follow a standardized diet for 1 week, followed by 3 weeks consumption of the same diet enriched with small amounts of different tomato products providing as a mean 8 mg lycopene, 0.5 mg β-carotene and 11 mg vitamin C per day. Plasma and lymphocyte concentrations of carotenoids, vitamin C were analysed.

Results: Dietary intervention with tomato products increased lycopene concentration both in plasma and lymphocytes. Vitamin C concentrations increased by 35% in plasma (P<0.05) and by 230% in lymphocytes (P<0.005).
Conclusion: Tomato products are not only good sources of lycopene but also sources of bioavailable vitamin C. A Regular intake of small amounts of tomato products can increase cell protection from DNA damage induced by oxidant species. This effect may originate from the synergism of different antioxidants present in tomatoes. (Riso P et al., Eur J Clin Nutr. 2004 October;58(10):1350-8)
Brief Description of the Structural Formula of Lutein, Lycopene, β-carotenE and Vitamin C
1. Lutein, molecular formula is: C₄₀H₅₆O₂, Its structural formula is shown:

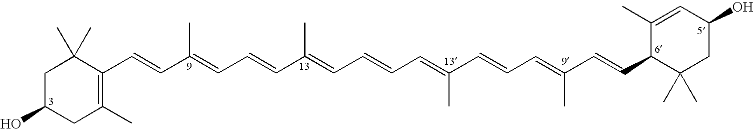

2. Lycopene, molecular formula is: C₄₀H₅₆, Its structural formula is shown:

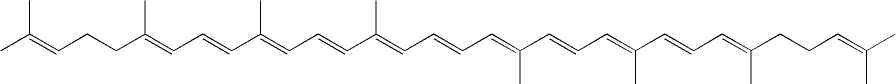

3. β-carotene, molecular formula is: C₄₀H₅₆, Its structural formula is shown:

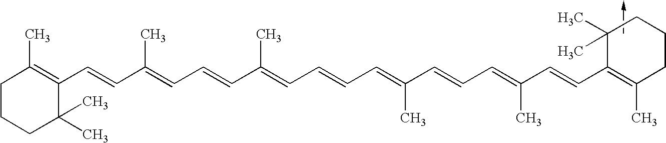

4. Vitamin C, molecular formula is: C₆H₈O₆, Its structural formula is shown:

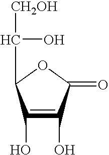

What is claimed is:

1. A mixture of antioxidants containing lipid-soluble carotenoids of β-carotene, lycopene, and lutein in various weight ratios, among which is 0.5:4:4, with Vitamin C added in weight ratio of 10, produced into powder or tablets as antioxidant drinks or food.

2. The mixture of antioxidants as claimed in claim 1, wherein said the rate of free radical scavenging activity per gram of mixed antioxidants is greater than the scavenging activity rate of 5.48×10⁴ ppm per gram of vitamin E alone.

3. The mixture of antioxidants as claimed in claim 1, wherein said the daily dietary intake should consist of 8.0 mg to 15.0 mg of Vitamin C, 0.3 mg to 0.8 mg of β-carotene, 2.0 mg to 8.0 mg of lycopene, and 2.0 mg to 8.0 mg of lutein.

4. The mixture of antioxidants as claimed in claim 1, wherein said antioxidants of β-carotene, lutein, lycopene and Vitamin C are produced by fine chemical synthesis or extraction from natural food, and treated with drying process into water-soluble or lipid-soluble powder, and then formulated into powder or tablets.

5. A manufacturing method for preparing the mixture of antioxidant compounds used in drinks or food, wherein said drinks for dissolving in 120 cc to 180 cc water without metal ions at room temperature between 15° C. and 25° C.
